(19) United States
(12) Patent Application Publication
(10) Pub. No.: US 2025/0002244 A1
(43) Pub. Date: Jan. 2, 2025

(54) APPARATUS, SYSTEM, AND METHOD FOR PACKAGING A BIOPROCESSING BAG

(71) Applicant: GLOBAL LIFE SCIENCES SOLUTIONS USA LLC, Marlborough, MA (US)

(72) Inventors: Deeyu CULLEN, Marlborough, MA (US); Wei JIN, Marlborough, MA (US); Benjamin ROBBINS, Marlborough, MA (US)

(73) Assignee: GLOBAL LIFE SCIENCES SOLUTIONS USA LLC, Marlborough, MA (US)

(21) Appl. No.: 18/699,939

(22) PCT Filed: Oct. 28, 2022
(86) PCT No.: PCT/US2022/048154

§ 371 Date: Apr. 10, 2024

Related U.S. Application Data
(60) Provisional application No. 63/273,301, filed on Oct. 29, 2021.

(51) Int. Cl. B65D 85/30; C12M 1/00

(52) U.S. Cl. CPC B65D 85/30; C12M 23/14

(57) ABSTRACT

Packaging for a bioprocessing bag includes a clam shell structure to receive a portion of the bag in a folded state, the bag having an impeller. The clam shell structure has a bottom shell and a top shell that mates with the bottom shell to define an interior portion with an opening extending therethrough to receive at least a portion of the folded bioprocessing bag and the impeller. The impeller, when placed in the packaging, is centrally disposed in the interior portion and a portion of the bioprocessing bag extends out from the opening of the clam shell structure. The bottom shell and the top shell each has a plurality of supporting features to secure and protect the bioprocessing bag and the impeller received in the clam shell structure from impact forces applied to the structure.

### BACKGROUND

### Technical Field

Embodiments of the invention relate generally to bioprocessing systems and methods and, more particularly, to an improved packaging for a flexible bioprocessing bag and associated components.

### Discussion of Art

A variety of vessels, devices, components and unit operations are known for carrying out biochemical and/or biological processes and/or manipulating liquids and other products of such processes. In order to avoid the time, expense, and difficulties associated with sterilizing the vessels used in biopharmaceutical manufacturing processes, single-use or disposable bioreactor bags and single-use mixer bags are used as such vessels. For instance, biological materials (e.g., animal and plant cells) including, for example, mammalian, plant or insect cells and microbial cultures can be processed using disposable or single-use mixers and bioreactors.

These single use or disposable bioprocessing bags are typically flexible or collapsible plastic bags that are supported by an outer rigid structure such as a stainless steel shell or vessel. Use of sterilized disposable bags eliminates the time-consuming step of cleaning of the vessel and reduces the chance of contamination. In operation, a bag may be positioned within the rigid vessel and filled with the desired fluid for mixing. An agitator assembly disposed within the bag is used to mix the fluid. Existing agitators are either top-driven (having a shaft that extends downwardly into the bag, on which one or more impellers are mounted) or bottom-driven (having an impeller disposed in the bottom of the bag that is driven by a magnetic drive system or motor positioned outside the bag and/or vessel). Most magnetic agitator systems include a rotating magnetic drive head outside of the bag and a rotating magnetic agitator (also referred to in this context as the “impeller”) within the bag. The movement of the magnetic drive head enables torque transfer and thus rotation of the magnetic agitator allowing the agitator to mix a fluid within the vessel.

The flexible, single use bioprocessing bags can vary in size from a few liters up to several thousand liters, and include the various ports, tubing, connectors, and other components mentioned above necessary to allow for mixing, sparging, draining, sampling and sensing/measurement of the contents of the bags. As will be appreciated, such bags are therefore quite large and cumbersome, and can be unwieldy to handle, transport, inspect and install.

Currently, existing bioprocessing bags and associated components are folded, wrapped, double-bagged, placed in a cardboard box, and filled with bubble wrap. This packaging may not provide adequate protection to the associated components of the bioprocessing bags such as the impeller. For example, the blades of the impeller are subject to damage that results from impact forces that can be applied to the packaging from multiple directions. The potential damage that can occur from impact forces applied to the packaging includes, but is not limited to, shearing of the blades of the impeller, cracking of the blades and total breakage of the blades.

### BRIEF DESCRIPTION

The various embodiments of the present invention provide an improved packaging for a bioprocessing bag that addresses the issues associated with conventional packaging.

In an embodiment, an apparatus for packaging for a bioprocessing bag having an impeller disposed within the bioprocessing bag is provided. The packaging includes a clam shell structure to receive a portion of the bioprocessing bag and the impeller in a folded state. The clam shell structure has a bottom shell and a top shell that mates with the bottom shell. The mated bottom shell and top shell define an interior portion with an opening extending therethrough to receive at least a portion of the folded bioprocessing bag and the impeller. The impeller, when placed in the packaging, is centrally disposed in the interior portion and a portion of the bioprocessing bag extends out from the opening of the clam shell structure. The bottom shell and the top shell each has a plurality of supporting features to secure and protect the bioprocessing bag and the impeller received in the clam shell structure.

In another embodiment, a packaging system including a clam shell structure having a bottom shell and a top shell that mates with the bottom shell is provided. The mated bottom shell and top shell define an interior portion with an opening extending therethrough. The system further includes a bioprocessing bag having an impeller disposed within the interior of the clam shell structure. The bioprocessing bag is in a folded state within the interior portion and the impeller is centrally disposed in the interior portion and a portion of the bioprocessing bag extends out from the opening of the clam shell structure. The bottom shell and/or the top shell has a plurality of supporting features to secure and protect the bioprocessing bag and the impeller.

### DRAWINGS

The present invention will be better understood from reading the following description of non-limiting embodiments, with reference to the attached drawings, wherein below:

FIG. 1 is a simplified side elevational, cross-sectional view of a bioprocessing bag having an impeller according to an embodiment of the present invention.

FIG. 2 is a perspective view of a clam shell structure packaging for a bioprocessing bag, like for example, the one depicted in FIG. 1, according to an embodiment of the present invention.

FIG. 3 is a perspective view showing more details of a top shell from the clam shell structure depicted in FIG. 2 according to an embodiment of the present invention.

FIG. 4 is a perspective view showing more details of a bottom shell from the clam shell structure depicted in FIG. 2 according to an embodiment of the present invention.

FIG. 5 is a perspective view of a clam shell structure packaging according to an alternative embodiment of the present invention.

FIG. 6 is a perspective view of a bottom shell from the clam shell structure of FIG. 5.

FIG. 7 is a perspective view of a top shell from the clam shell structure of FIG. 5.

FIG. 8 is a perspective view of a clam shell structure and outer packaging, e.g., box, according to an embodiment of the invention.

FIG. 9 is a perspective view of a clam shell structure and bag according an embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, or a rigid container, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems. As used herein, the term “bag” means a flexible or semi-rigid container or vessel used, for example, as a bioreactor or mixer for the contents within.

Embodiments of the present invention are directed to an improved packaging for a bioprocessing bag that provides solutions to the technical problems associated with conventional bag packaging. In one embodiment, a packaging for a bioprocessing bag having an impeller is provided. The packaging comprises a clam shell structure to receive the bioprocessing bag and the impeller in a folded state. The clam shell structure has a bottom shell and a top shell that mates with the bottom shell. The mated bottom shell and top shell define an interior portion with an opening extending therethrough to receive at least a portion of the folded bioprocessing bag and the impeller. The impeller, when placed in the packaging, is centrally disposed in the interior portion and a portion of the bioprocessing bag extends out from the opening of the clam shell structure. The bottom shell and the top shell each has a plurality of supporting features to secure and protect the bioprocessing bag and the impeller received in the clam shell structure.

Although the embodiments of the present invention are described with respect to packaging for a bioprocessing bag such as a single-use, flexible bioreactor bag for use in bioprocessing operations, it is contemplated that the packaging disclosed herein may likewise be utilized for packaging and transporting flexible bags for use in a variety of industries and end uses.

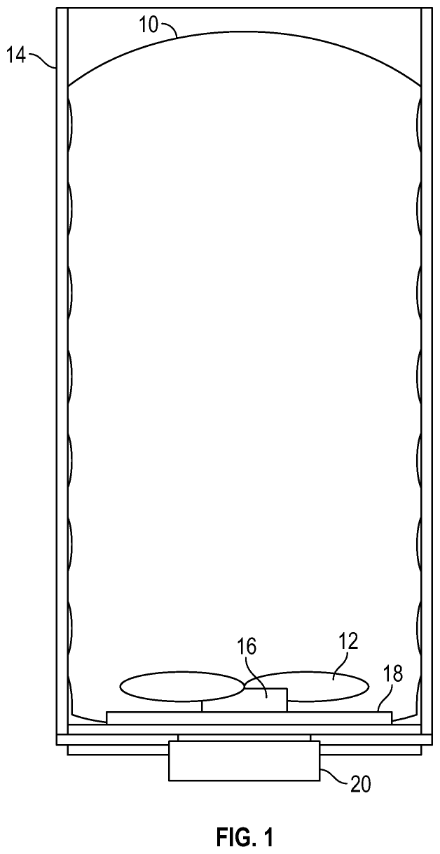

Turning now to the figures, FIG. 1 shows a simplified side elevational, cross-sectional view of a bioprocessing bag 10 having an impeller 12 according to an embodiment of the present invention. As shown in FIG. 1, the bioprocessing bag 10, which can include a single-use, flexible bag, is disposed within a rigid shell or vessel 14 and restrained thereby.

In one embodiment, the single-use, flexible bioprocessing bag 10 can be formed of a suitable flexible material, such as a homopolymer or a copolymer. The flexible material can be one that is USP Class VI certified, for example, silicone, polycarbonate, polyethylene, and polypropylene. Non-limiting examples of flexible materials include polymers such as polyethylene (for example, linear low-density polyethylene and ultra low-density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics.

In an embodiment, the flexible material may be a laminate of several different materials. Portions of the flexible container can comprise a substantially rigid material such as a rigid polymer, for example, high density polyethylene, metal, or glass. The flexible bag may be supplied pre-sterilized, such as using gamma irradiation.

As shown in FIG. 1, the bioprocessing bag 10 contains the impeller 12. In one embodiment, the impeller 12 can be attached to a magnetic hub 16 at the bottom center of the inside of the bag 10, which rotates on an impeller plate 18 also positioned on the inside bottom of the bag 10. Together, the impeller 12 and the hub 16 (and in some embodiments, the impeller plate 18) form an impeller assembly. A magnetic drive 20 external to the vessel 14 provides the motive force for rotating the magnetic hub 16 and the impeller 12 to mix the contents of the bioprocessing bag 10. While FIG. 1 illustrates the use of a magnetically driven impeller, other types of impellers and drive systems are also possible, including top-driven impellers.

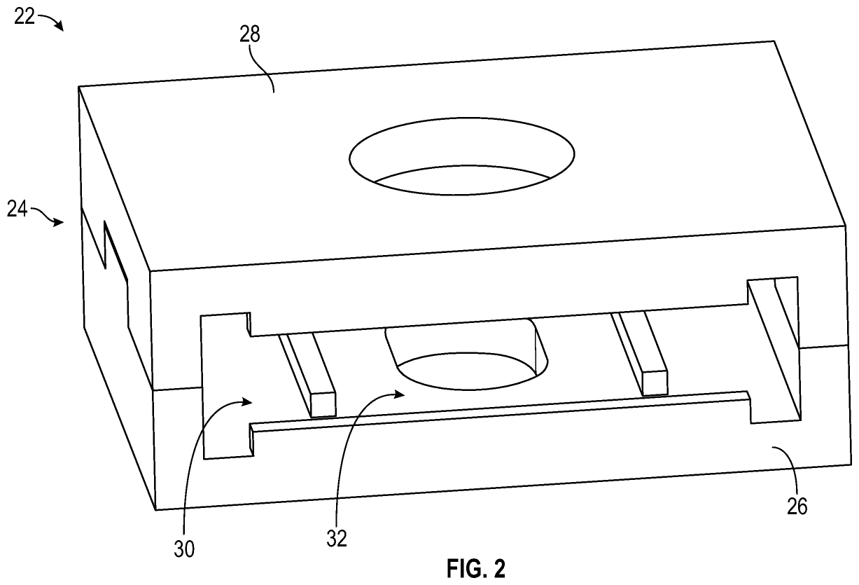

As indicated, pre-sterilized, single-use flexible bags such as for example, the bioprocessing bag 10 depicted in FIG. 1, can be large and cumbersome to handle, transport, inspect and install. Embodiments of the present invention as depicted in FIGS. 2-4 provide a packaging apparatus 22 for the bioprocessing bag 10 and associated components such as the impeller 12 after folding and wrapping thereof.

The packaging 22 as shown in FIG. 2, includes a clam shell structure 24 to receive the bioprocessing bag 10 and the impeller 12 (both shown in FIG. 1) in a folded state. The clam shell structure 24 includes a bottom shell 26 and a top shell 28 that mates with the bottom shell. The mated bottom shell 26 and top shell 28 define an interior portion 30 with an opening 32 extending therethrough to receive at least a portion of the folded bioprocessing bag and the impeller. As shown, the packaging 22 has a substantially quadrilateral or rectangular shape, though in certain embodiments the shape may vary.

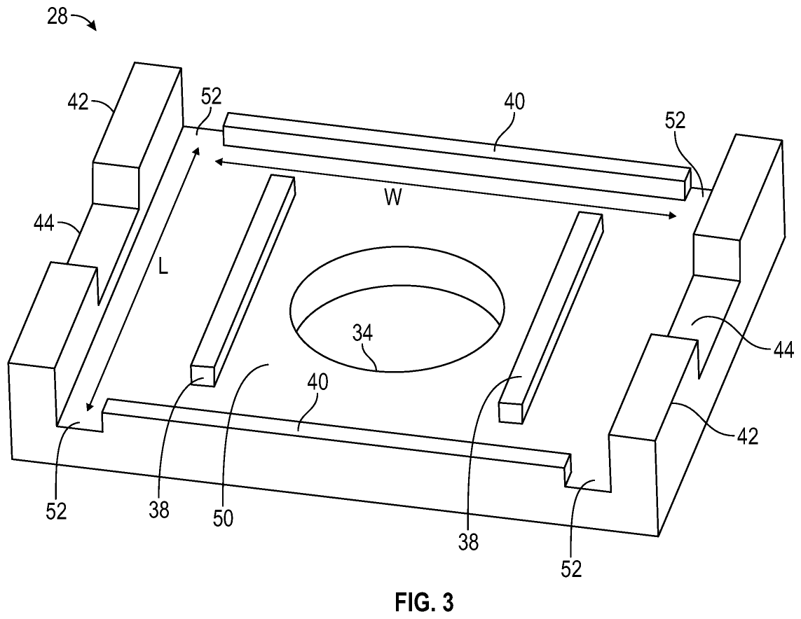

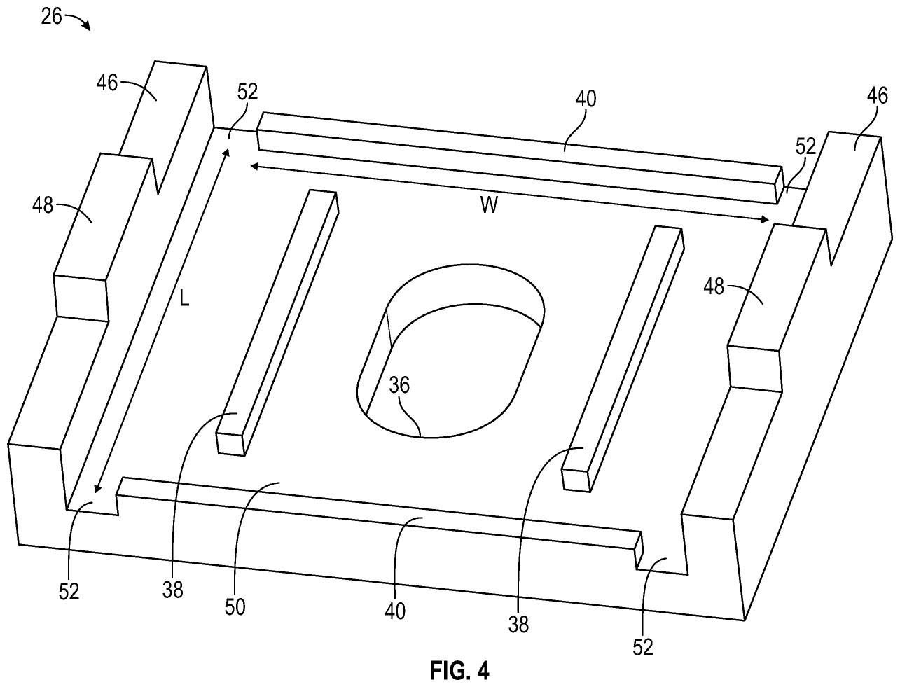

In one embodiment, the impeller is centrally disposed in the interior portion 30 and a portion of the bioprocessing bag can extend out from the opening 32 of the clam shell structure 24. Although not shown in FIG. 2, the bottom shell 26 and the top shell 28 each has a plurality of supporting features, e.g., ribs, to secure and protect the bioprocessing bag and the impeller received in the clam shell structure 24. Further details of the bottom shell 26 and the top shell 28, including the plurality of supporting features, are shown in FIGS. 3 and 4.

FIGS. 3 and 4 are perspective views that show more details of the top shell 28 and the bottom shell 26, respectively, from the clam shell structure 24 depicted in FIG. 2, according to embodiments of the present invention. As shown in these figures, the bottom shell 26 and the top shell 28 each comprises a cutout centrally disposed about the interior portion 30 of the clam shell structure 24. In particular, FIG. 3 shows the top shell 28 having round-shaped cutout 34, while FIG. 4 shows the bottom shell 26 having an elongated-shaped (e.g., ovular or elliptical) cutout 36.

To this extent, the cutout 34 of the top shell 28 allows room for a portion of the impeller to protrude in instances where an impact is applied to the clam shell structure 24 that may cause movement of the impeller towards the top shell 28. The cutout 36 of the bottom shell 26 is elongated to provide a wider opening as blades of the impeller 12 and other of its components may shift due to impact to the claim shell structure 24 that can occur through the handling and transporting of the packaging 22.

It understood that the shapes of the cutouts 34 and 36 are illustrative of several possible shapes and are not meant to limit the embodiments of the present invention as it is contemplated that other shapes can be used to provide similar predetermined margins of movement between the bottom shell and the top shell in response to impact forces (e.g., static and dynamic loads) applied to the claim shell structure that may arise during handling and transport of the packaging 22.

As noted above, the top shell 28 and the bottom shell 26 have a plurality of supporting features to secure and protect the bioprocessing bag 10 and the impeller 12 while received in the clam shell structure 24. In one embodiment, the plurality of supporting features comprises a pair of centrally disposed opposing ribs/supporting features 38 extending substantially across a length (L) of the bottom shell 26 and the top shell 28 and a pair of raised edge portions/side disposed supporting features 40 extending substantially across the width (W) of the bottom shell 26 and the top shell 28.

The pairs of centrally disposed opposing supporting features 38 provide nearby support to the impeller when placed thereabout. In particular, the centrally disposed opposing supporting features 38 restrict movement of the impeller and bag when encapsulated by the clam shell structure 24. The pairs of side disposed supporting features 40, which are substantially perpendicular to the centrally disposed supporting features 38, provide a degree of far support to the impeller when placed thereabout. To this extent, the centrally disposed supporting features 38 provide relaxation to the restriction in movement of the impeller when encapsulated by the clam shell structure 24.

Although the supporting features 38, 40 are shown as linear, rectangular ribs, in embodiments, the shape may vary without departing from the scope of the invention. For example, in certain embodiments, the features 38, 40 may be non-linear, e.g., curved, and may have non-rectangular cross-sections. Likewise, the spatial relationship between the features 38, 40 may, in certain embodiments, depart from the depicted relationship.

The supporting features 38, 40 may be manufactured from the same material as the clam shell structure 24, e.g., a rigid plastic. In this regard, features 38, 40 may be molded into the shells 26, 28. In other embodiments, the features 38, 40 may be separate structures that are adhered or affixed to the shells.

In one embodiment, the top shell 28 and the bottom shell 26 can be mated together using a tongue and groove joint configuration. As shown in FIG. 3, the top shell 28 can have a pair of side walls/opposing edge features 42 each having grooves 44 extending across the length L of the top shell, while FIG. 4 shows the bottom shell 26 having a pair of side walls/opposing edge features 46 each having tongue structures 48 extending across the length L of the bottom shell. In this manner, the plurality of tongue structures 48 of the bottom shell 26 can be correspondingly received and mated with the plurality of grooves 44 of the top shell 28. As will be appreciated, the tongue structures 48 and grooves 44 are secured together/mated via a press/interference fit.

Although embodiments of the clam shell structure 24 are depicted and described as being joined together via tongue structures 48 and grooves 44 that are integral to the clam shell structure 24, other mechanical and/or chemical connection mechanisms may be employed such as latches, clamps, adhesives, hook and loop fasteners, without departing from the scope of the invention.

In one embodiment, upon coupling of the top shell 28 with the bottom shell 26, the plurality of supporting structures 38, 40 of the bottom shell and the top shell can be in vertical alignment with each other and located in the claim shell structure 24 between the mated plurality of grooves 44 and the plurality of tongue structures 48. For example, the pairs of centrally disposed opposing supporting features 38 and the pair of side disposed supporting features 40 are positioned between the mated tongue and groove coupling of the top shell 28 and the bottom shell 26. With this configuration, the plurality of supporting structures 38 and 40 of the bottom shell 26 and the top shell 28 is separated from the mated plurality of grooves 44 and the plurality of tongue structures 48 by a predetermined spacing 50, which refers to the substantially flat open space around the centrally disposed supporting features 38 that is bordered/delineated by the edge features 42 and side disposed supporting features 40

The predetermined spacing is configured to provide clearance to receive the folded bioprocessing bag. For example, the spacing 50 allows the folded bioprocessing bag to relax into the interior portion 30. FIGS. 3 and 4 also show that the predetermined spacing 50 includes spacing extensions 52 formed between the pairs of side disposed supporting features 40 of the top shell 28 and the bottom shell 26 and the pair of opposing edge features 42 of the top shell and the pair of opposing edge features 46 of the bottom shell 26. This spacing provides room for the bag edges to be tucked into.

While embodiments are depicted and described with each of the top and bottom shells 28, 26 having a plurality of supporting structures, it may be possible that, in certain embodiments, only one of the top and bottom shells includes a plurality of supporting structures. It's also possible that one of the top or bottom shells 28, 26 includes a single supporting structure, while the opposite shell includes a plurality.

Similarly, embodiments may be used with bags 10 that include impellers of various sizes, shapes, and blade configurations. By way of non-limiting examples, bags having impellers with a diameter of 7.5 inches (19.05 cm) and 8.7 inches (22.09 cm) may be packaged using the inventive clam shell structure 24.

In embodiments, the clam shell structure 24 can be manufactured from a variety of materials including rigid plastics or other durable materials that are impact resistant and suitably lightweight.

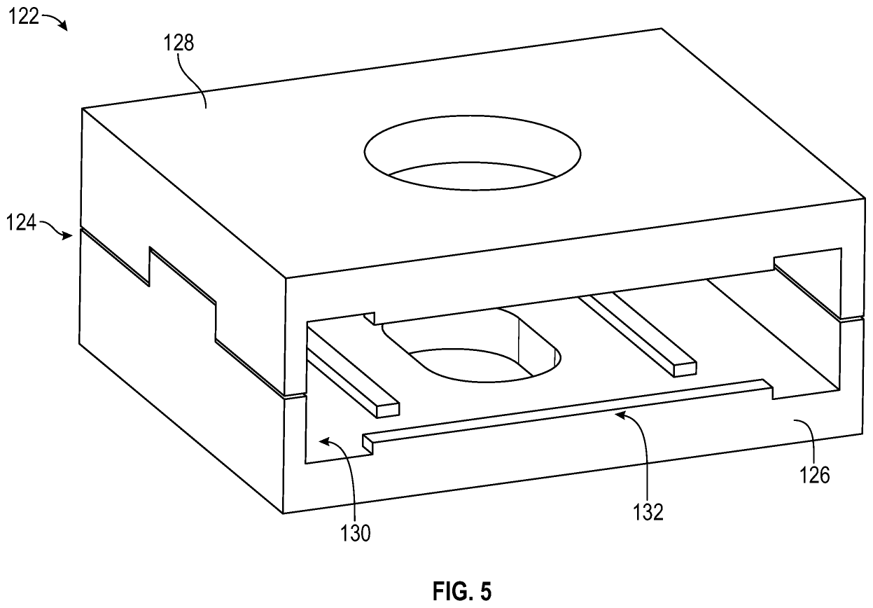

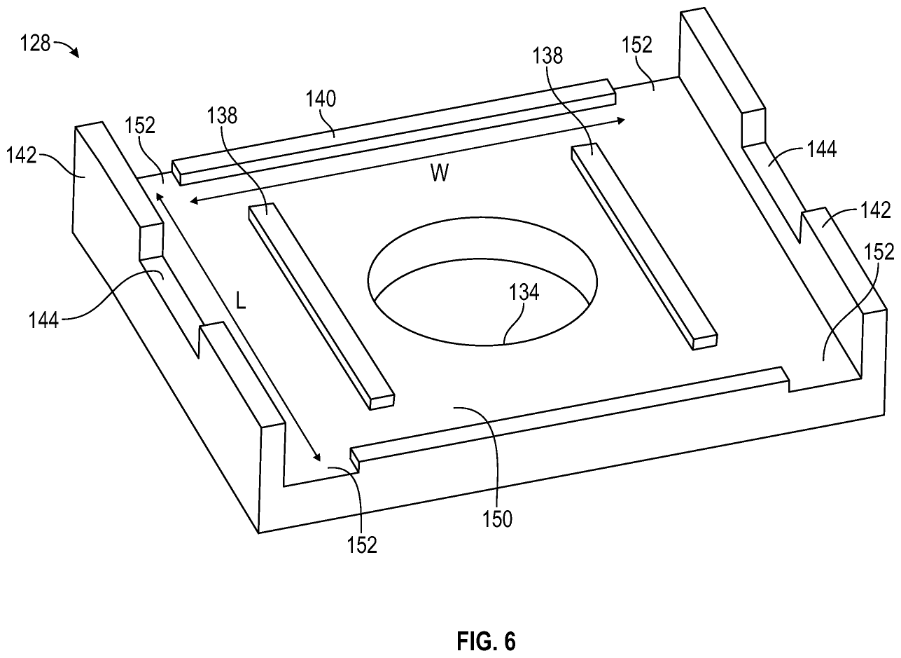

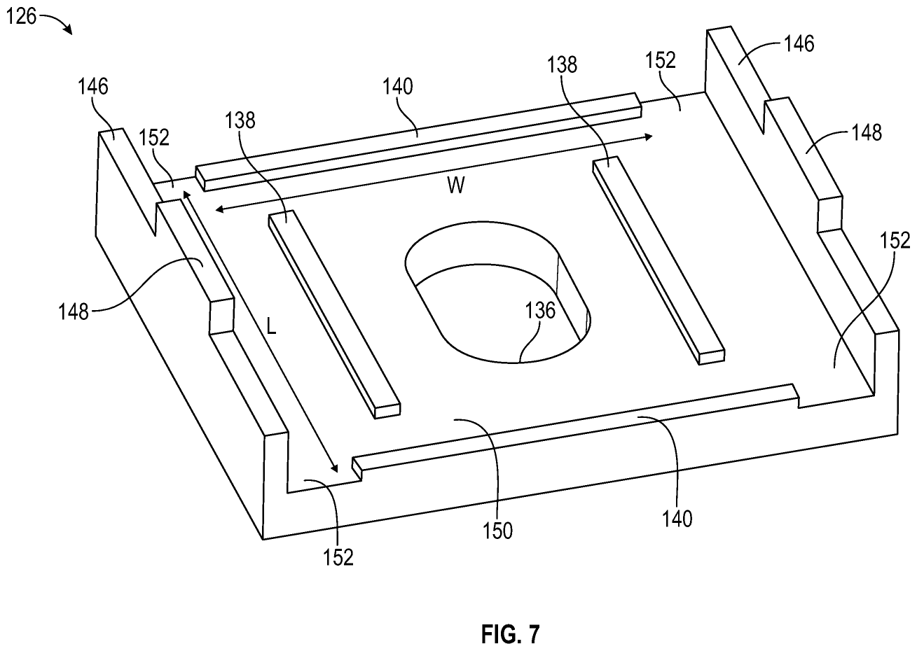

Referring now to FIGS. 5-7, an embodiment that is particularly suitable for sterilization via conventional sterilization equipment and for use with external packaging is depicted. Much like the embodiment of FIGS. 2-4, the packaging apparatus 122 includes a clam shell structure 124 having a top shell 128 and a bottom shell 126. The top and bottom shells 128, 126 include centrally disposed and side disposed supporting features 138, 140 (extending across a length L and width W of the shells respectively) and opposing edge features 142, 146. The edge features 142, 146, include a groove 144 and tongue structure 148, which serve as a mechanism for securing the top shell 128 to the bottom shell 146. The top shell 128 includes a cut out 134 that is substantially round and the bottom shell 126 includes an elongated cut out 136.

As with the aforementioned embodiment, the top and bottom shells 128, 126 are joined together to form an interior portion 130 with an opening 132 that extends through the apparatus 122. The interior portion 130 includes a predetermined spacing 150 (e.g., the substantially flat open space around the centrally disposed supporting features 138 that is bordered/delineated by the edge features 142 and side disposed supporting features 140). The predetermined spacing 150 includes spacing extensions 152 which accommodate edge portions of the folded bag.

Notably, the side walls/edge features 142, 146 are thinner, e.g., approximately 1 inch (2.54 cm) thick, and the ribs/centrally disposed supporting features 138 and raised edge portion/side disposed support features 140 are approximately half the height of their counterparts in the embodiment of FIGS. 2-4, with an approximate height of ½ inch (1.27 cm). As will be appreciated, this creates a larger interior portion 130 and a larger opening 132.

In a specific embodiment, the overall clam shell structure 24/124 has an approximate length of 22 inches (55.88 cm), a width of 26 inches (66.04 cm), and a height of about 9 inches (22.86 cm). In such embodiments, the ribs/centrally disposed supporting features 38/138 are about 14 inches (35.56 cm) in length and the raised edge portions/side disposed support features 40/140 are about 18 inches (45.72 cm) in length. The groove 44/144 is about 7 inches (17.78 cm) long and the tongue 48/148 is about 6.74 inches (17.12 cm) long. The top shell cut out 34/134 has a diameter of about 9 inches (22.86 cm) and the spacing extensions 152 are about 3 inches (7.62 cm) wide.

Such embodiments may be particularly suitable for packaging and transport of 1000 L flexible bioreactor bags. As will be appreciated, the dimensions of the structure 24/124 may vary depending upon the size of the bag/vessel 10 to be received in the structure 24. Embodiments may also be sized and configured for use with 50 L, 100 L, 200 L, and 500 L bags.

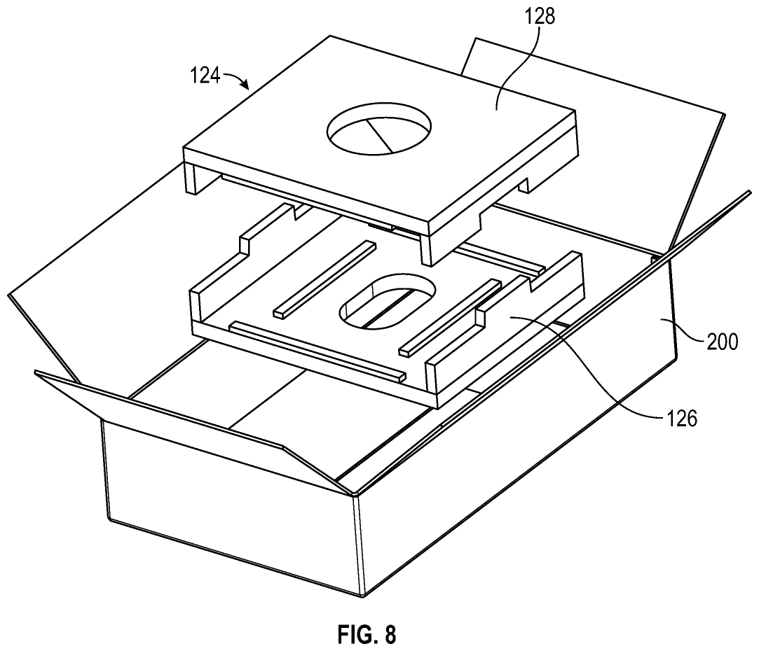

Referring now to FIG. 8, in certain embodiments, the clam shell structure 124 is configured to be placed within a rectangular box 200, e.g., cardboard box, for shipping purposes. As shown, the box 200 is longitudinally longer than the clam shell structure 124 so that the entire bag (not shown) will fit within the box 200. To facilitate removal of the structure 124 from such a box 200, the exterior of the structure 24 may include indents or grooves or other handle structures (not shown). The clam shell structure 124 may also be sterilized, e.g., with gamma radiation, prior to use.

In use, a folded bioprocessing vessel/bag 10 is placed on a bottom shell 26/126 of the clam shell structure 24/124 with the impeller portion of the bag 10 is placed over the elongated-shaped (e.g., ovular or elliptical) cutout 36/136 in the bottom shell 26/126. The top shell 28/128 is then placed on top of the bottom shell 26/126 and secured to the bottom shell 26/126, via, for example, a tongue and groove attachment mechanism, so that the impeller of the vessel is centrally disposed in an interior portion 30/130 of the clam shell structure 24/124. The bioprocessing bag 10 and impeller are secured and protected by a plurality of supporting features located on the bottom shell 26/126 and/or the top shell 28/128.

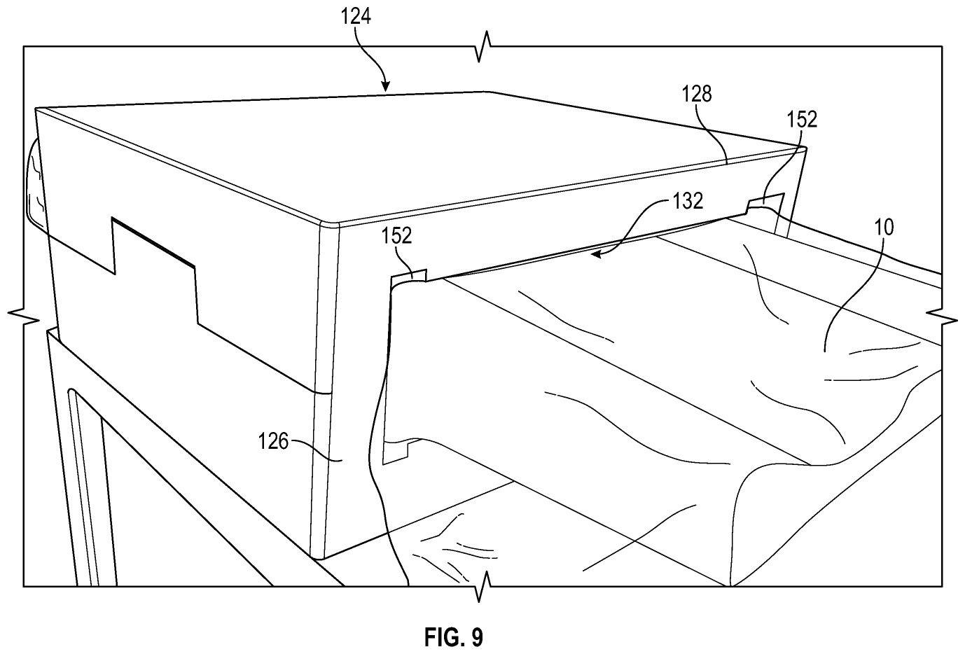

FIG. 9 depicts a folded bag 10 within the clam shell structure 124. As shown, the bag 10 protrudes through the opening 132 at both the front and back of the shell structure 124. Spacing extensions 152 accommodate edge portions of the folded bag 10.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

In the appended claims, the terms “including” and “in which” are used as the plain-English equivalents of the respective terms “comprising” and “wherein.”Moreover, in the following claims, terms such as “first,” “second,” “third,” “upper,” “lower,” “bottom,” “top,” etc. are used merely as labels, and are not intended to impose numerical or positional requirements on their objects.

What is claimed is:

1. An apparatus for packaging a bioprocessing bag having an impeller disposed within the bioprocessing bag comprising:
a clam shell structure to receive a portion of the bioprocessing bag and the impeller in a folded state, the clam shell structure including:
a bottom shell;
a top shell that mates with the bottom shell, the mated bottom shell and top shell defining an interior portion with an opening extending therethrough to receive at least a portion of the folded bioprocessing bag and the impeller; and
wherein the impeller, when placed in the packaging, is centrally disposed in the interior portion and a portion of the bioprocessing bag extends out from the opening of the clam shell structure; and
wherein the bottom shell and the top shell each has a plurality of supporting features to secure and protect the bioprocessing bag and the impeller received in the clam shell structure.

2. The apparatus according to claim 1, wherein the bottom shell and the top shell each comprises a cutout centrally disposed about the interior portion of the clam shell structure.

3. The apparatus according to claim 2, wherein the cutout of the bottom shell is elongated, and the cutout of the bottom shell is round.

4. The apparatus according to claim 1, wherein the plurality of supporting features comprises a pair of centrally disposed opposing supporting features extending substantially across a length of the bottom shell and the top shell, and a pair of side disposed supporting features extending substantially across a width of the bottom shell and the top shell.

5. The apparatus according to claim 1, wherein the bottom shell comprises a plurality of tongue structures and the top shell comprises a plurality of grooves to correspondingly receive and mate with the plurality of tongue structures to secure the top and bottom shells together.

6. The apparatus according to claim 5, wherein the plurality of supporting structures of the bottom shell and the top shell are located in the claim shell structure between the mated plurality of grooves and the plurality of tongue structures.

7. The apparatus according to claim 6, wherein the plurality of supporting structures of the bottom shell and the top shell are separated from the mated plurality of grooves and the plurality of tongue structures by a predetermined spacing, wherein the predetermined spacing is configured to provide clearance to receive parts of the folded bioprocessing bag.

8. The apparatus according to claim 1, wherein the plurality of supporting features provides the impeller with a predetermined margin of movement between the bottom shell and the top shell in response to an impact force applied to the claim shell structure.

9. A packaging system comprising:
a clam shell structure including:
a bottom shell;
a top shell that mates with the bottom shell, the mated bottom shell and top shell defining an interior portion with an opening extending therethrough;
a bioprocessing bag having an impeller disposed within the interior portion of the clam shell structure; and
wherein bioprocessing bag is in a folded state within the interior portion, the impeller is centrally disposed in the interior portion, and a portion of the bioprocessing bag extends out from the opening of the clam shell structure; and
wherein the bottom shell and/or the top shell has a plurality of supporting features to secure and protect the bioprocessing bag and the impeller.

10. The packaging system according to claim 9 wherein the bottom shell and the top shell each comprises a cutout centrally disposed about the interior portion of the clam shell structure.

11. The packaging system according to claim 10, wherein the cutout of the bottom shell is elongated, and the cutout of the bottom shell is round.

12. The packaging system according to claim 9, wherein the plurality of supporting features comprises a pair of centrally disposed opposing supporting features extending substantially across a length of the bottom shell and the top shell, and a pair of side disposed supporting features extending substantially across a width of the bottom shell and the top shell.

13. The packaging system according to claim 9, wherein the bottom shell comprises a plurality of tongue structures and the top shell comprises a plurality of grooves to correspondingly receive and mate with the plurality of tongue structures to secure the top and bottom shells together.

14. The packaging system according to claim 13, wherein the plurality of supporting structures of the bottom shell and the top shell are located in the claim shell structure between the mated plurality of grooves and the plurality of tongue structures.

15. The packaging system according to claim 14, wherein the plurality of supporting structures of the bottom shell and the top shell are separated from the mated plurality of grooves and the plurality of tongue structures by a predetermined spacing, wherein the predetermined spacing is configured to provide clearance to receive parts of the folded bioprocessing bag.

16. The packaging system according to claim 9, wherein the plurality of supporting features provides the impeller with a predetermined margin of movement between the bottom shell and the top shell in response to an impact force applied to the claim shell structure.
